(19) United States
(12) Patent Application Publication
(10) Pub. No.: US 2009/0270612 A1
(43) Pub. Date: Oct. 29, 2009

(54) Therapeutic Agent for Osteoporosis Using a Retinoid X Receptor-Related Compound

(76) Inventors: Nobuyuki Udagawa, Nagano (JP); Midori Nakamura, Nagano (JP); Hiroyuki Kagechika, Tokyo (JP)

Correspondence Address: BAKER BOTTS L.L.P., 2001 ROSS AVENUE, SUITE 600, DALLAS, TX, 75201-2980, US

(21) Appl. No.: 12/065,994

(22) PCT Filed: Sep. 4, 2006
(86) PCT No.: PCT/JP2006/317454

§ 371 Date: Mar. 6, 2008

(30) Foreign Application Priority Data
Sep. 6, 2005 (JP) 2005-258480

(51) Int. Cl. C07D 243/10

(52) U.S. Cl. 540/555

(57) ABSTRACT

It is an object of the present invention to provide a novel therapeutic agent for osteoporosis.

HX531 is utilized, which is synthesized as a retinoid X receptor antagonist and has an activity in inhibiting a differentiation into adipocytes. Furthermore, any dosage form containing HX531 as an effective component can be used.

### CROSS-REFERENCE TO RELATED APPLICATION

This application claims priority to PCT/JP2006/317454, entitled A Therapeutic Agent for Osteoporosis Using a Retinoid X Receptor-Related Compound, filed Sep. 4, 2006, which claims priority to JP 2005-258480 filed Sep. 6, 2005.

### TECHNICAL FIELD OF THE INVENTION

The present invention relates to a therapeutic agent of a retinoid X receptor-related compound for osteoporosis, and more particularly, it relates to a novel use of HX531 synthesized as a retinoid X receptor antagonist for the treatment of osteoporosis.

### BACKGROUND OF THE INVENTION

Osteoporosis cases have been increasing with the rapid spread of an aging population. In osteoporosis, the most serious and the most burdensome complication is a fracture. A transcervical fracture and a vertebral fracture cause a bedridden condition, thereby decreasing the quality of life (QOL) greatly and increasing the social and economical burden imposed by out-patient care or hospitalization. Therefore, prevention of and treatment for osteoporosis are important tasks to accomplish for the aging population, which will expand in the future. Recent studies of bone metabolism have been conducted dramatically against the background of these social needs.

Bones play important roles in storing calcium as well as supporting the body, protecting organs, and making hemocytes in bone marrow. Strictly speaking, bones consist of a bone matrix composed of a protein called collagen and bone minerals composed of calcium, phosphorus, and magnesium. Bones undergo metabolism as well as other cells and are maintained by a balance of cells breaking bones (hereinafter referred to as osteoclasts) and cells forming bones (osteoblasts).

In addition, calcium, an important component for bones, is an essential element for life support. Referring to the role of bones, storing calcium, as the calcium concentration decreases in a serum, the osteoclasts dissolve calcium from the bones into the serum to keep a constant concentration of calcium and maintain physiological functions. Thus, the calcium concentration in serum is strictly regulated.

Osteoporosis occurs when the function of breaking bones exceeds the function of forming them. That is the condition called osteoporosis, where collagen or calcium, a component of bones, is decreased and the bones undergo rarefaction. Especially in females after menopause, a secretion of estrogens which protects their bones is decreased. Consequently, the efficiency of the osteoblasts in forming bones is decreased while the activity of the osteoclasts in bone resorption is increased, thereby enhancing the possibility of developing the condition of osteoporosis.

Hence, it is of the utmost importance to maintain a balance of the osteoblasts and the osteoclasts for improving the condition of osteoporosis.

There are now a calcitonin and a bisphosphonate used as medicines to repress the activity of the osteoclasts in bone resorption.

Calcitonins inhibit bone resorption highly by binding to a receptor which is expressed specifically in the osteoclasts. However, there is a disadvantage that a continuous administration of calcitonins causes a loss of the calcitonin receptors (down-regulation), decreasing the effect of the calcitonins. Furthermore, a medical examination by a doctor is needed for each administration since formulation of the calcitonins is only in the form of injection. Then, it is difficult to undergo a long-term administration of calcitonins.

On the other hand, bisphosphonates suppress bone resorption highly by being incorporated into the osteoclasts. However, it is pointed out that there is a possibility of causing gastrointestinal disorders such as esophagitis or a gastric ulcer as a side effect of taking bisphosphonates. It is considered that an allowable acidic condition in gastric mucosa as well as the osteoclasts is to convert bisphosphonates from aqueous (polar) to lipophilic (nonpolar) such that the bisphosphonates can be incorporated easily into gastric parietal cells.

Also, since the bisphosphonate has a characteristic of being deposited on bones, other than the above side effect, it has been considered as a problem that there is a possibility of causing an inhibitory action on a normal coupling mechanism between bone resorption and bone formation by a long-term accumulation of the bisphosphonates.

Therefore, with the advancing of the aging population, it is desired to develop a medicine which acts on osteoporosis more effectively, requires fewer administrations, and has fewer side effects.

Patent Document 1 Japanese Laid-Open Patent Application 2003-526677

Patent Document 2 Japanese Laid-Open Patent Application 2002-539106

Patent Document 3 Japanese Laid-Open Patent Application 2003-531180

Patent Document 4 Japanese Laid-Open Patent Application 2003-519103

### SUMMARY OF THE INVENTION

A knockout mouse has been produced which lacks a gene for an osteoprotegerin (OPG), which is an inhibitor for bone resorption. Since this knockout mouse does not have an inhibitor for bone resorption, more osteoclasts have been found than in a normal mouse and bone resorption is proceeded actively. Therefore, bone resorption is remarkably facilitated with age, counted in weeks, thereby causing serious osteoporosis.

In an osteoporotic patient and an OPG-KO mouse having a condition of osteoporosis, markedly more fat is realized in a bone marrow. An undifferentiated mesenchymal cell, in bone tissues, is differentiated into chondroblasts, osteoblasts, and adipocytes. In the pathology of osteoporosis, after bone resorption is effected by osteoclasts, an undifferentiated mesenchymal cell is differentiated only into adipocytes and the resorbed parts of the bone are considered to be replaced by adipose tissues.

From this point of view, the inventors conducted a large-scale experiment with an object to analyze a differentiation and a bone resorption mechanism of the osteoclast which is the only type of cell performing bone resorption. As a result, based on a finding that the adipocytes in the resorbed parts of the bone are not only a space filler but also a fate of the mesenchymal cell which cannot be differentiated into osteoblast, the present invention has been developed by an inference that if it is possible to differentiate the undifferentiated mesenchymal cell into osteoblasts instead of adipocytes, bones can be regenerated, resulting in a treatment for osteoporosis.

Accordingly, the present invention has been made on the basis of the above, and it is an object of the present invention to provide a use of HX531 as a novel therapeutic agent for osteoporosis, which is synthesized as a retinoid X receptor antagonist and has an activity in inhibiting a differentiation into adipocytes.

That is, the above object can be accomplished by a therapeutic agent for osteoporosis containing a retinoid X receptor-related compound as claimed in claim 1.

According to the invention as claimed in claim 1, the retinoid X receptor-related compound can be provided for use as a novel therapeutic agent for osteoporosis.

The invention as claimed in claim 2 is characterized in that in the invention claimed in claim 1, said retinoid X receptor-related compound is HX531.

According to the invention as claimed in claim 2, HX531 can be provided for use as a novel therapeutic agent for osteoporosis, which is synthesized as a retinoid X receptor antagonist and has an activity in inhibiting a differentiation into adipocytes.

In accordance with the invention, HX531 can be utilized as a therapeutic agent for enhancement of local bone resorption observed in a systemic metabolic bone disease similar to osteoporosis, periodontitis, or rheumatoid arthritis. HX531 of the invention is synthesized as a nuclear receptor retinoid X receptor antagonist having an activity in inhibiting a differentiation into adipocytes. HX531 is also applicable to an antidiabetic or anti-obesity agent.

### BRIEF DESCRIPTION OF THE DRAWINGS

FIG. 1 is a graph showing bone mass in a lumbar trabecular bone by bone morphometry (osteogenic marker).

FIG. 2 is a graph showing trabecula numbers in a lumbar trabecular bone by bone morphometry (osteogenic marker).

FIG. 3 is a graph showing osteoid mass in a lumbar trabecular bone by bone morphometry (osteogenic marker).

FIG. 4 is a graph showing osteoblast surface (bone surface) in a lumbar trabecular bone by bone morphometry (osteogenic marker).

FIG. 5 is a graph showing a bone calcification rate in a lumbar trabecular bone by bone morphometry (osteogenic marker).

FIG. 6 is a graph showing a bone formation rate in a lumbar trabecular bone by bone morphometry (osteogenic marker).

FIG. 7 is a graph showing a bone resorption rate in a lumbar trabecular bone by bone morphometry (bone resorption marker).

FIG. 8 is a graph showing osteoclast surface in a lumbar trabecular bone by bone morphometry (bone resorption marker).

FIG. 9 is a graph showing osteoclast number (bone surface) in a lumbar trabecular bone by bone morphometry (bone resorption marker).

FIG. 10 is a graph showing resorption surface in a lumbar trabecular bone by bone morphometry (bone resorption marker).

FIG. 11 is a graph showing rarefaction surface/cortical bone in a femoral cortical bone by bone morphometry.

FIG. 12 is a graph showing porous surface in a femoral cortical bone by bone morphometry.

FIG. 13 is a graph showing a bone formation rate (endosteum surface/year) in a femoral cortical bone by bone morphometry.

FIG. 14 is a graph showing a bone formation rate (periosteum surface/year) in a femoral cortical bone by bone morphometry. FIG. 17 is withdrawn.

FIG. 15 is a graph showing osteoid surface in periosteum surface in a femoral cortical bone by bone morphometry.

FIG. 16 is a graph showing resorption surface/periosteum surface in a femoral cortical bone by bone morphometry.

### DETAILED DESCRIPTION OF THE INVENTION

In the following, a preferred aspect of the present invention will be described in detail, but the present invention is not limited to this aspect.

First, a nuclear receptor will be described prior to describing the invention.

A nuclear receptor is a transcription factor which a low molecular weight compound such as a steroid hormone or lipophilic vitamin binds to and activates in order to regulate a specific gene expression. It has been suggested recently that the nuclear receptor is involved in the onset of various diseases such as cancer, diabetes, or arteriosclerosis and considered as an important molecular target in drug development.

HX531 is a retinoid X receptor-related compound, and more particularly, it is synthesized as a retinoid X receptor (hereinafter referred to as RXR) antagonist. Since the possibility was discovered that the decline in an activity of RXR led to a treatment for diabetes and obesity from an experiment using a mouse, the application of HX531 as a medicine has been noted.

A nuclear receptor is, generally, a substance working as a transcription factor when a ligand binds to the receptor to form a complex which further attaches to DNA. The first discovered nuclear receptor was a steroid hormone receptor, and in the mid-1980s, its gene was cloned to sequence, and it has been discovered that homology between the receptor and DNA is high. Thereafter progress in gene cloning and sequence analysis with homology of the sequence being used as a key has revealed many proteins similar to a nuclear receptor. In a recent human genome decoding, the number of those receptor-like proteins reached 48, which proteins are called a nuclear receptor superfamily.

A receptor is a protein found in a cell which recognizes a physical/chemical stimulus to cause the cell to respond. Transmitters including drugs are generically called a ligand or a first messenger. This ligand is considered as a “key” to transmit a signal while the receptor is considered as a “keyhole.” So, by an interaction of the “key” and the “keyhole”, it is recognized that the signal which the receptor relays controls development and morphogenesis through cell division, growth, and death control as well as is involved deeply in higher levels of biological phenomena such as perception, recognition, and movement.

Now, the function of a cell will be described to understand the receptor.

A human cell consists of a cell membrane, cytoplasm, and a nucleus.

Other than said receptor, an ion channel, which is responsible for activities including regulation of ion balance in and outside the cell, cell excitation, and secretion, is found in the cell membrane. Some of the receptors are combined with ion channels (ion channel-coupled receptors), which transmit a signal inside the cell using an electric potential generated by a passage of the ions.

In cytoplasm, there is a unit such as a ribosome, Goldi body, mitochondrion, and endoplasmic reticulum for protein synthesis, energy production, and all kinds of signal transductions whereas a gene (DNA) is stored in a nucleus. It is a protein such as an enzyme that is involved in regeneration of body tissues and progress of chemical reactions in the body. A protein is composed of amino acids. After information which determines a sequence of those amino acids is read from a gene (DNA) to a messenger RNA (transcription), a required protein is synthesized in a ribosome.

Receptors are divided into two categories, a nuclear receptor (also called an intracellular receptor) and a membrane (-penetrating) receptor, based on where they are found.

A nuclear receptor accepts a lipophilic substance such as a lipophilic vitamin A and D, a steroid hormone, and a thyroid hormone that can pass freely through the cell membrane. These ligands (transmitters) bind the nuclear receptor to a specific sequence of DNA in a nucleus to transcribe DNA, and a required protein is synthesized to cause a biological reaction.

Particularly, a peroxisome proliferator-activated receptor (PPAR) of a steroid hormone receptor family has been lately noted as a transcription factor which regulates various target genes involved in a glucose/lipid metabolism. A ligand is bound to PPAR to form a heterodimer with a retinoid X receptor (RXR), and the heterodimer is bound to a PPAR response element found upstream on the target DNA to control the transcription.

Many PPARα are found in a fat-consuming organ including the liver, heart, kidney, and brown adipocyte, PPARβ in the brain, and PPARγ in a fat-storing organ including a white adipocyte. For example, a fibrate of a lipid-lowering agent acts as a ligand for PPARα and has multifunctional effects such as improvement of lipid, antiinflammation, and antioxidation. Furthermore, PPARγ acts as a transcription factor involved in an adipocyte differentiation by combining with RXR and increases the number of small adipocytes to improve insulin resistance. A thiazolidine derivative is known as a ligand for PPARγ.

On the other hand, a membrane (-penetrating) receptor accepts an aqueous substance on the membrane surface such as a peptide hormone, neurotransmitter, and growth factor which cannot pass through the cell membrane. After the acceptance, the membrane receptor performs its function by transmitting a signal indirectly through a transmitter in the cytoplasm called a second messenger. Finally, the receptor prompts the suppression of an activity of substances in the body and the transcription of a specific gene (DNA) found in a cell nucleus to synthesize a protein which causes a biological reaction.

Now, a structure and a property of the receptor will be described.

As mentioned above, a receptor is composed of a combination of amino acids (protein) which is a “keyhole” having a complicated tertiary structure. When a ligand fits the keyhole, it is considered to have an affinity for the receptor. Even a chemical compound which is not originally found in vivo can affect a cell since it has a common tertiary structure with a true ligand in vivo (intrinsic ligand) and there is an affinity for the receptor.

The number of keyholes (receptors) can vary dynamically based on the environment in the cell. Therefore, if necessary, the number of the receptors can be decreased (down-regulation) or increased (up-regulation).

Next, a correlation between the selectivity of the ligand (transmitter) and a drug will be described.

There is a ligand with low selectivity which fits different types of keyholes like a master key while there is a ligand with high selectivity which fits only specific types of keyholes. Drugs can be divided into a receptor stimulating agent or receptor blocker from the viewpoint of drugs acting on the receptors.

A drug (key) acting on a keyhole (receptor) to transmit an original signal to a cell is called a receptor stimulating agent (agonist). On the other hand, there is a drug (key) called a receptor blocker (antagonist) which inhibits the binding of a true ligand to a receptor by fitting a specific keyhole.

In the present invention, HX531 synthesized as an RXR antagonist is used as an RXR-related compound to repress a differentiation into an adipocyte and enhance a differentiation into an osteoblast. Although an antagonist binds antagonistically to a receptor for a bioactive substance such as a hormone, it is a substance which does not have any bioactive effect through a receptor. Drugs and pollutants (endocrine-disrupting chemicals) which are antagonists have a molecular structure similar to a “key” of a hormone and bind to the receptors, but they do not show any bioactive effect through the receptor. Consequently, those antagonists inhibit a binding of the hormone to the receptor, repressing the original effect of the hormone.

Even though HX531 is a compound synthesized by the present inventors which can be applied to an antidiabetic/anti-obesity agent, in the present invention, it is effective as a therapeutic agent for osteoporosis.

Therefore, for a therapeutic agent of the invention, medical compositions can be utilized, containing HX531 as an effective component which is synthesized as a nuclear receptor RXR antagonist, and the therapeutic agent can be synthesized with ease.

In the present invention, a dosage form of a therapeutic agent for osteoporosis with medical compositions containing HX531 as an effective component, which is a nuclear receptor RXR antagonist, is not limited in particular and can be selected properly, if necessary. By way of example, HX531 is available for clinical application as an oral medicine including tablets, capsules, granule, fine granule, and powder or an injection.

In this case, although the dose will vary depending on age, sex, weight, and severeness of the disease of osteoporosis patients, it may be typically in a range of 20 mg to 2 g per day per adult and preferably administered in several portions.

For a conventional treatment for osteoporosis, a calcitonin and a bisphosphonate have been used as drugs which repress an activity of the osteoclasts in bone resorption. However, ingestion of HX531 in accordance with the present invention, which is a RXR-related compound and synthesized as RXR antagonist, acts effectively on a bone formation mechanism of the osteoblasts and does not cause a side effect such as a gastrointestinal disorder by a bisphosphonate or nonresponsiveness of a calcitonin. Then, HX531 can be utilized effectively as a therapeutic agent for osteoporosis.

As described above, in a treatment for osteoporosis which has been difficult to provide, HX531 can be a novel drug to regenerate bones since the administration of a compound containing HX531 as an effective component, which is a RXR-related compound and synthesized as RXR antagonist, can differentiate an undifferentiated mesenchymal cell into not only an adipocyte but also an osteoblast.

### EXAMPLES

In the following, specific examples in accordance with the present invention will be described in detail, but the invention is not limited to these examples. Various aspects are available for the details within the spirit and scope of the invention.

### Example 1

OPG knockout mice (6 week-old) were divided into 3 groups with 7 mice being prepared for each group. These mice were obtained from CLEA Japan, Inc. and categorized into three groups: control group (Cont), HX531 group (HX), and LE 135 group (LE). LE135 is a compound synthesized as a nuclear receptor retinoid A receptor (RAR) antagonist.

Paste feed is prepared by adding sterile water into and softening sterilized pellets in a dish. HX531 group and LE135 group were provided with the paste feed in which a corresponding drug was mixed. The amount of feed provided per day was fixed at 3 g per mouse in a solid state. The amount of drugs was calculated at 0.03% to the feed weight. Although the paste feed was exchanged everyday, almost no feed was left in all groups. A drug was administered successively for 4 weeks, and mice were double-labeled with tetracyclines and calceins by an abdominal administration 4 days and 2 days before they were sacrificed.

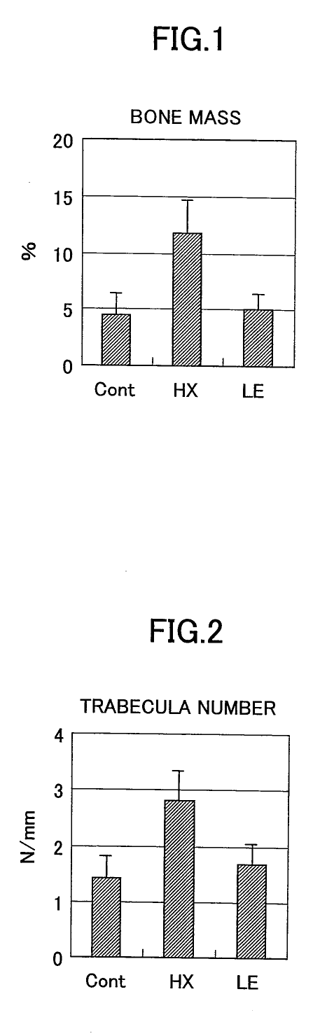

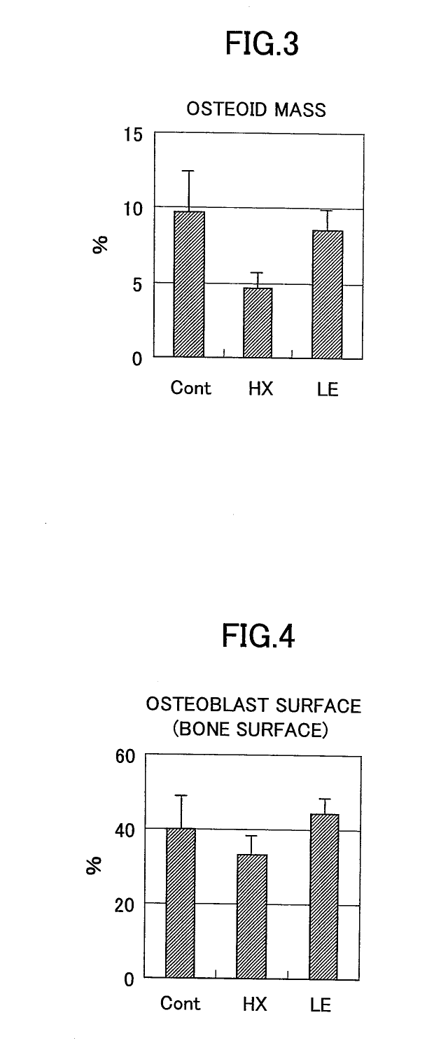

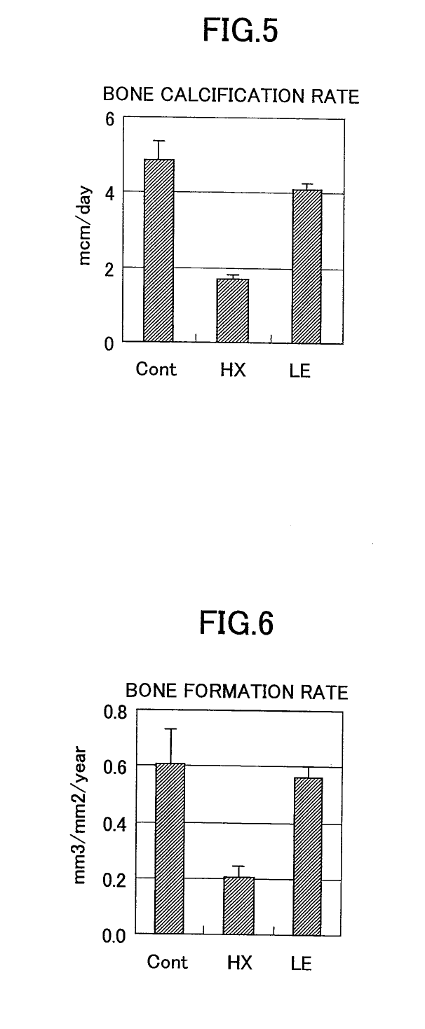

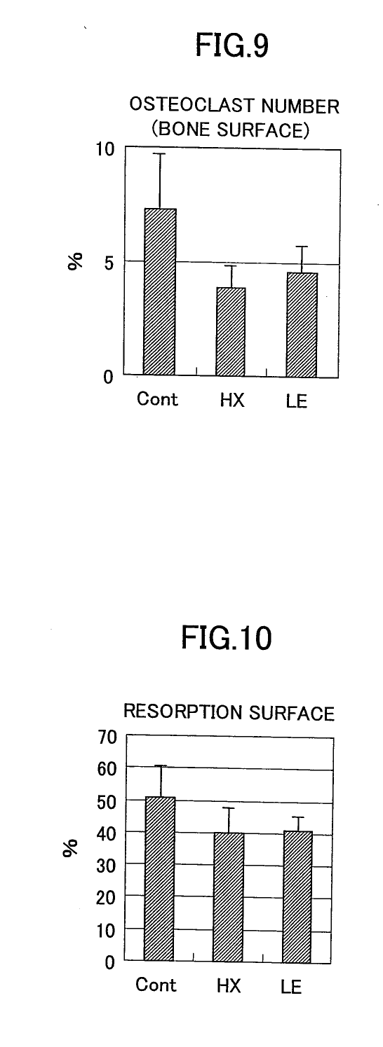

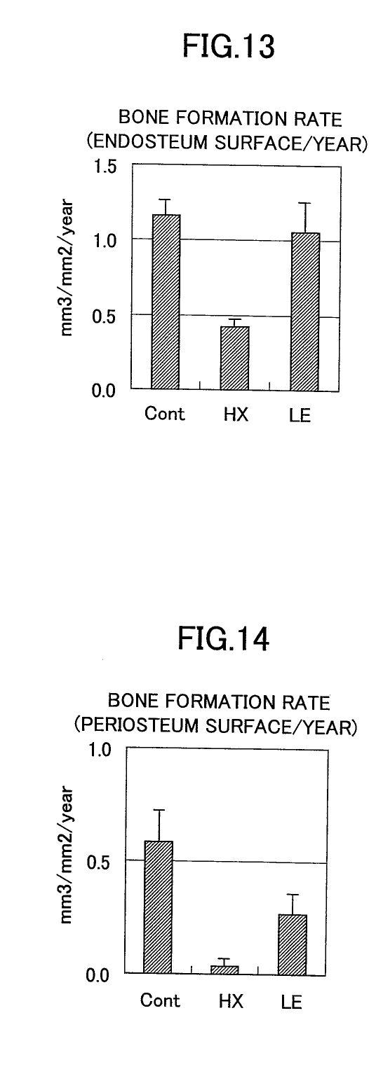

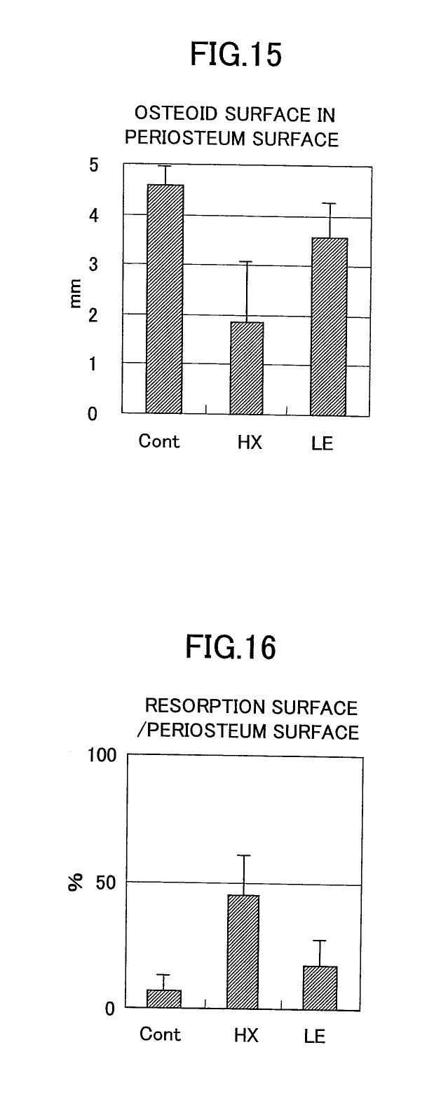

While bone morphometry of femur (cortical bone) and lumbar (trabecular bone) was conducted, a histological observation of femur was made. Furthermore, a blood sample was taken before the sacrifice to measure a bone formation marker. The results are shown in FIG. 1 to 16.

FIGS. 1 through 6 are the figures showing bone mass, trabecula number, osteoid mass, osteblast surface (bone surface), bone calcification rate, and a bone resorption rate, respectively, in a lumbar trabecular bone by bone morphometry (bone formation marker). As found in these figures, especially in FIG. 1 and FIG. 2, the amount of bone mass and trabecula number in the mice provided with HX531 were large compared to the mice from the control group or mice provided with LE135, meaning that HX531 is effective in treating osteoporosis.

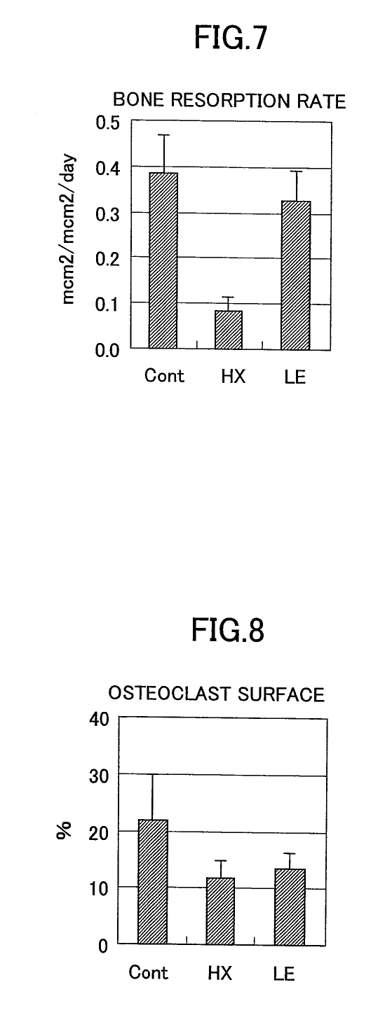

FIGS. 7 through 10 are the figures showing a bone resorption rate, osteoclast surface, osteoclast number (bone surface), resorption surface, multinuclear osteoclast number, and mononuclear osteoclast number, respectively, in a lumbar trabecular bone by bone morphometry (bone formation marker). As found in these figures, especially in FIG. 7 and FIG. 8, a bone resorption rate of the mice provided with HX531 was significantly low and the amount of osteoclast surface in the mice from HX531 group was small compared to the mice from the control group, meaning that HX531 is effective in treating osteoporosis.

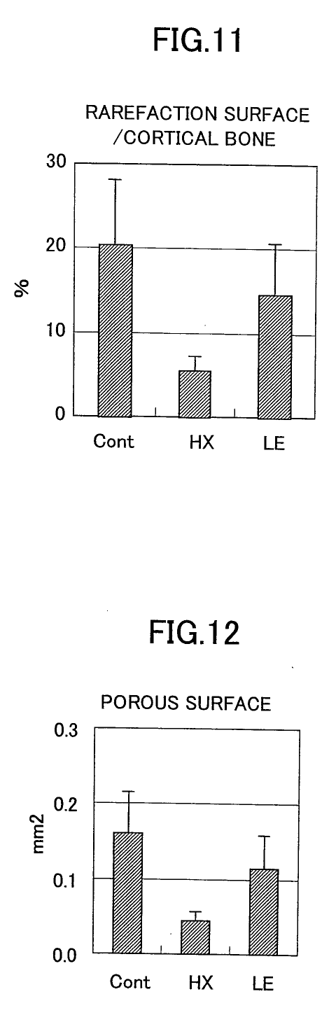

FIGS. 11 through 16 are the figures showing rarefaction surface/cortical bone, porous surface, a bone formation rate (endosteum surface/year), (periosteum surface/year), osteoid surface in periosteum surface, and resorption surface/periosteum surface in a femoral cortical bone by bone morphometry. As found in these figures, especially in FIG. 11, the amount of rarefaction surface/cortical bone of the mice provided with HX531 was significantly low compared to the mice from the control group, and further in FIG. 12, the amount of porous surface is small, meaning that HX531 is effective in treating osteoporosis.

Although the preferred examples of the invention have been described above, the invention is not limited to these specific embodiments. Different variations and modifications may be made within the spirit and scope of the invention as defined in the claims.

### INDUSTRIAL APPLICABILITY

According to the present invention, it is confirmed that problems of the conventional therapeutic agent for osteoporosis such as 1) decline in a function of calcitonin caused by loss of calcitonin receptors, 2) gastrointestinal disorder caused by acceleration of taking bisphosphonates in under acid conditions, 3) long-term deposition of bisphosphonates in bone tissues, are resolved in an animal experiment by an oral administration of HX531 to a mouse, which is synthesized as a retinoid X receptor antagonist and has an activity in inhibiting a differentiation into adipocytes. Therefore, HX531 can be newly utilized as a therapeutic agent for osteoporosis and is available as an oral medicine.

The present application is based on Japanese priority application No. 2005-258480 filed on Sep. 6, 2005, with the Japanese Patent Office, the entire contents of which are hereby incorporated by reference.

What is claimed is:

1. A therapeutic agent for osteoporosis, comprising a retinoid X receptor-related compound.

2. The therapeutic agent for osteoporosis as claimed in claim 1, characterized in that said retinoid X receptor-related compound is HX531.
